(19) United States
(12) Patent Application Publication
(10) Pub. No.: US 2018/0319835 A1
(43) Pub. Date: Nov. 8, 2018

(54) METHOD OF PREPARING NUCLEASE-RESISTANT DNA-INORGANIC HYBRID NANOFLOWERS

(71) Applicants: KOREA ADVANCED INSTITUTE OF SCIENCE AND TECHNOLOGY, Daejeon (KR); Gachon University of Industry-Academic cooperation Foundation, Gyeonggi-do (KR)

(72) Inventors: Hyun Gyu Park, Daejeon (KR); Moon Il Kim, Gyeonggi-do (KR); Ki Soo Park, Daejeon (KR); Chang Yeol Lee, Daejeon (KR)

(21) Appl. No.: 15/924,242

(22) Filed: Mar. 18, 2018

(30) Foreign Application Priority Data
May 2, 2017 (KR) 10-2017-0056226

(51) Int. Cl. C07H 23/00; C07H 1/00

(52) U.S. Cl. CPC C07H 23/00; C07H 1/00

(57) ABSTRACT

A method of preparing nucleic acid-inorganic hybrid nanoflowers is described, in which a nucleic acid is allowed to react with a solution of a metal ion-containing compound at room temperature, thereby forming a complex between the metal ion and the nitrogen atom of an amide bond or amine group present in the nucleic acid. Organic-inorganic hybrid nanoflower structures thus may be synthesized using nucleic acid in a simple manner under an environmentally friendly condition without any toxic chemical substance. The produced organic-inorganic hybrid nanoflower structures exhibit a high DNA encapsulation yield, nuclease resistance, and significantly increased peroxidase activity. These nanoflower structures may be widely used as gene therapy carriers and in biosensing technology.

### CROSS-REFERENCE TO RELATED APPLICATION

The priority under 35 USC § 119 of Korean Patent Application 10-2017-0056226 is hereby claimed. The disclosure of Korean Patent Application 10-2017-0056226 is hereby incorporated herein by reference, for all purposes.

### TECHNICAL FIELD

The present invention relates to a method of preparing nuclease-resistant DNA-inorganic hybrid nanoflowers, and more particularly to a method of preparing nucleic acid-inorganic hybrid nanoflowers, which comprises allowing a nucleic acid to react with a solution of a metal ion-containing compound at room temperature, thereby forming a self-assembled complex between the metal ion and the nitrogen atom of an amide bond or amine group present in the nucleic acid, which resembles flower in nanometer scale.

### BACKGROUND ART

Flower-shaped nanomaterials called nanoflowers have attracted attention in various fields, including catalysis, electronics and analytical chemistry, due to their property of having a rough surface and a large surface-to-volume ratio (A. Mohanty et al., Angew. Chem. Int. Ed. 2010, 5, 4962; J. Xie et al., ACS Nano 2008, 23, 2473; Z. Lin, Y et al., RSC Adv., 2014, 4, 13888). Recently, the Zare research group succeeded in synthesizing organic/inorganic hybrid nanoflowers using various enzymes and proteins with copper sulfate at room temperature, and found that enzymes loaded on the hybrid nanoflowers have higher activity, stability and durability than general enzymes dissolved in aqueous solutions (J. Ge et al., Nanotechnol., 2012, 7, 428). This increased enzymatic activity may be applied to systems that analyze various materials in a highly sensitive and stable manner. Until now, biosensor systems for the detection of phenol, hydrogen peroxide and glucose have been developed (L. Zhu et al., Chem. Asian. J., 2013, 8, 2358; Z. Lin et al., ACS. Appl. Mater. Inter., 2014, 6, 10775; J. Sun et al., Nanoscale, 2014, 6, 255).

A protein that forms the nanoflowers contains many nitrogen atoms in the amide bonds and amine groups, and a possible synthetic mechanism was proposed according to which such moieties form complexes with copper ions via coordination interaction, so that the synthesis of primary copper-protein nanoparticles will be induced, and consequently, nanoflower structures will be formed by time-dependent precipitation (J. Ge, et al., Nat. Nanotechnol., 2012, 7, 428). For example, it was found that organic-inorganic hybrid nanoflowers can be synthesized using various proteins such as bovine serum albumin, a-lactalbumin, laccase, carbonic anhydrase, and lipase (B. S. Batule et al., J. Nanomedicine, 2015, 10, 137). The above-described technology is meaning significant in that it is a new technology of synthesizing nanoflower structures using proteins. However, its expansion to other organic biological molecules has not been reported.

Accordingly, the present inventors have found that nucleic acid incubated with a metal ion-containing compound can induce a hybrid nanoflower, which consists of both nucleic acid and metal compound as organic and inorganic compound, respectively. The incubation is performed at room temperature under an environmentally friendly condition in a very simple manner, and the nucleic acid-inorganic hybrid nanoflowers thus produced have low cytotoxicity, show significantly increased loading capacities compared to those produced by a conventional DNA loading technology, and have high resistance against nuclease, thereby completing the present invention.

### SUMMARY OF INVENTION

It is an object of the present invention to provide a method of preparing DNA-inorganic hybrid nanoflowers, comprising synthesizing nucleic acid-inorganic hybrid nanoflowers in a very simple manner at room temperature under an environmentally friendly condition without addition of any toxic reducing agents or the like.

Another object of the present invention is to provide nucleic acid-inorganic hybrid nanoflowers that have low cytotoxicity, show high loading capacities, and have high resistance against nuclease.

The above objects of the present invention can be achieved by the present invention as specified below.

To achieve the above objects, the present invention provides a method of preparing nucleic acid-inorganic hybrid nanoflowers, comprising forming a complex between a metal ion and a nitrogen atom of an amide bond or amine group in the nucleic acid, by reacting the nucleic acid with a solution of the metal ion-containing compound at room temperature.

The present invention also provides nucleic acid-inorganic hybrid nanoflowers having resistance against nuclease, which are produced by the above-described method.

The present invention also provides a carrier for gene therapy, which comprises the above-described nucleic acid-inorganic hybrid nanoflowers.

The present invention also provides a biosensor comprising the above-described nucleic acid-inorganic hybrid nanoflowers.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a process of synthesizing organic-inorganic hybrid nanoflowers using DNA according to an example of the present invention (FIG. 1a), and depicts SEM images showing time-dependent formation of nanoflower structures (FIG. 1b).

FIG. 2 depicts SEM images showing the effect of the DNA concentration on the formation of nanoflower structures according to an example of the present invention.

FIG. 3 depicts SEM images showing the effect of the DNA nucleotide sequence and length on the formation of nanoflower structures according to an example of the present invention.

FIG. 4 depicts electrophoresis images showing the results of analyzing whether DNA loaded on nanoflower structures is degraded by DNase I (FIG. 4A) and exonuclease III (FIG. 4B), which are nucleases, according to an example of the present invention.

FIG. 5 is a graph showing cytotoxicity test results for DNA-inorganic hybrid nanoflower structures synthesized according to an example of the present invention.

FIG. 6 is a graph showing the results of analyzing the peroxidase activity of DNA-inorganic hybrid nanoflower structures synthesized according to an example of the present invention.

### BEST MODE FOR CARRYING OUT THE INVENTION

Unless defined otherwise, all the technical and scientific terms used herein have the same meaning as those generally understood by one of ordinary skill in the art to which the invention pertains. Generally, the nomenclature used herein and the experiment methods, which will be described below, are those well-known and commonly employed in the art.

In the present invention, it was found that when a nucleic acid was allowed to react with a solution of a metal ion-containing compound at room temperature, thereby forming a complex between the metal ion and the nitrogen atom of an amide bond or amine group present in the nucleic acid, nucleic acid-inorganic hybrid nanoflowers could be obtained which had resistance against nuclease and in which the DNA loaded on the nanostructures stably maintained its structure even after 24 hours of a sufficient enzymatic reaction.

Therefore, in one aspect, the present invention is directed to a method of preparing nucleic acid-inorganic hybrid nanoflowers, comprising forming a complex between a metal ion and a nitrogen atom of an amide bond or amine group in the nucleic acid, by reacting the nucleic acid with a solution of the metal ion-containing compound at room temperature.

In addition, in another aspect, the present invention is directed to nucleic acid-inorganic hybrid nanoflowers having resistance against nuclease, which are produced by the above-described method.

Based on the principle of synthesis of protein-based nanoflower structures, the present inventors have paid attention to the fact that nucleic acid which is another biopolymer substance also contains many amide bonds and amine groups, and the present inventors expected that the nucleic acid would induce flower-shaped nanostructures, similar to the protein. Based on this expectation, the present inventors carried out experiments, and as a result, have found for the first time that it is possible to synthesize organic-inorganic hybrid nanoflower structures using nucleic acid at room temperature under an environmentally-friendly condition in a very simple manner (K. S. Park, B. S. Batule, K. S. Kang, T. J. Park, M. I. Kim and H. G. Park, J. Mater. Chem. B, 2017, 5, 2231).

The present invention has the following advantages over a conventional method (D. Nykypanchuk et al., Nature, 2008, 451, 553) which uses DNA merely as a linker and only as a template for nanomaterial synthesis: 1) it is possible to synthesize nanoflower structures under an environmentally-friendly condition without addition of any toxic reducing agents; 2) the synthesized DNA-based nanoflower structures have low cytotoxicity; 3) DNA loaded on the nanoflower structures shows significantly increased loading capacities of 95% or more compared to those produced by a conventional DNA loading technology (K. E. Shopsowitz et al., Small, 2014, 10, 1623); and 4) DNA loaded on the nanoflower structures has high resistance against nuclease.

A method of preparing nucleic acid-inorganic hybrid nanoflowers according to the present invention and the nucleic acid-inorganic hybrid nanoflowers produced by the method will be described in detail hereinafter.

In the present invention, the nucleic acid may be DNA or RNA. The metal may be at least one selected from the group consisting of copper (Cu), zinc (Zn), calcium (Ca), and manganese (Mn), and preferably copper is used as the metal, but is not limited thereto.

In addition, in the present invention, the metal ion-containing compound may be at least one selected from the group consisting of copper sulfate (CuSO₄), zinc acetate (Zn(CH₃COO)₂), calcium chloride (CaCl₂), and manganese sulfate (MnSO₄), and preferably copper sulfate is used as the metal ion-containing compound, but is not limited thereto.

In the method of preparing nucleic acid-inorganic hybrid nanoflowers according to the present invention, the reaction may be performed at room temperature for 60 to 80 hours, and the concentration of the nucleic acid may be 10 pM to 100 μM, preferably 10 pM to 1 μM, depending on the length of the nucleotide sequence thereof. The size of the nucleic acid-inorganic hybrid nanoflowers may be determined depending on the concentration of the nucleic acid.

Further, in the present invention, it was found that the nucleic acid-inorganic hybrid nanoflowers can be utilized in biosensing technology for high-sensitivity detection of target biomaterials as well as can be utilized as a carrier for gene therapy as having no cytotoxicity.

Therefore, in still another aspect, the present invention is directed to a carrier for gene therapy and a biosnesor, which comprises the above-described nucleic acid-inorganic hybrid nanoflowers.

The DNA-inorganic hybrid nanoflower structures produced according to the present invention have no cytotoxicity (100% cell viability), and may be utilized as a carrier for gene therapy in the future. In addition, the DNA-inorganic hybrid nanoflower structures show high peroxidase activity due to their specific large surface area. Furthermore, the DNA-inorganic hybrid nanoflower structures have higher peroxidase activity than that of conventional protein-based nanoflower structures. Thus, the DNA-inorganic hybrid nanoflower structures synthesized according to the present invention may be widely utilized in biosensing technology for high-sensitivity detection of target biomaterials in the future.

### EXAMPLES

Hereinafter, the present invention will be described in further detail with reference to examples. It will be obvious to a person having ordinary skill in the art that these examples are for illustrative purposes only and are not to be construed to limit the scope of the present invention.

### Example 1: Production of Nuclease-Resistant DNA-Inorganic Hybrid Nanoflowers

DNAs having various nucleotide sequences and lengths were allowed to react with copper sulfate at room temperature for 3 days, thereby producing DNA-inorganic hybrid nanoflowers.

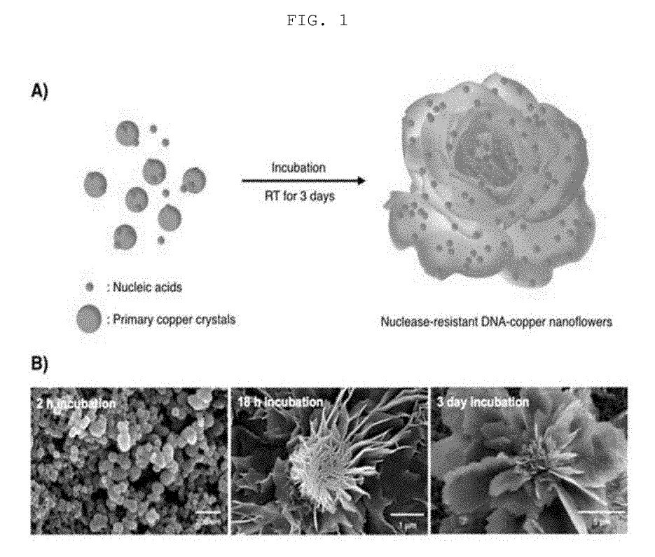

FIG. 1(a) shows a process of synthesizing organic-inorganic hybrid nanoflower structures using DNA. When DNAs having various nucleotide sequences and lengths are allowed to react with copper sulfate at room temperature for 3 days, nanoflower structures having large surface areas are obtained. In the principle of synthesis, the nitrogen atoms in amide bonds or amide groups present in the nucleic acids form a complex with copper ions, similar to proteins, whereby flower-shaped structures are synthesized. FIG. 1(b) depicts SEM (scanning electron microscope) images showing the time-dependent formation of nanoflower structures. As can be seen in FIG. 1(b), in the initial reaction stage (2 hours), small flower bud shapes were formed, and with the passage of time (18 hours), flower shapes were formed, and finally after 3 days, complete flower-shaped structures having a large surface-to-volume ratio were formed. This process of forming DNA-based nanoflower structures is similar to a process of forming nanoflowers using protein (J. Ge et al., Nanotechnol., 2012, 7, 428).

### Example 2: Production of DNA-Inorganic Hybrid Nanoflowers Using Various Concentrations of DNA

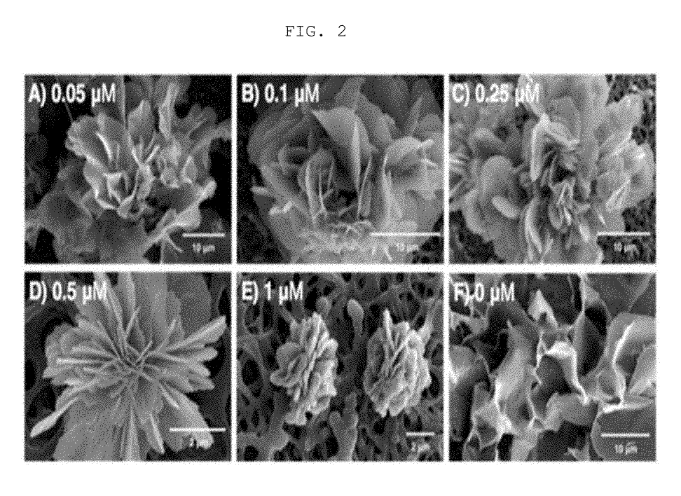

The effect of the DNA concentration on the production of DNA-inorganic hybrid nanoflowers was examined. FIG. 2 depicts SEM images showing the results of an experiment performed to examine the effect of the DNA concentration (A: 0.05 μM, B: 0.1 μM, C: 0.25 μM, D: 0.5 μM, E: 1 μM, F: 0 μM) on the formation of nanoflower structures. As can be seen in FIG. 2, when the DNA concentration was low, relatively large nanoflower structures having an average size of about 30 μm were formed (FIGS. 2A, 2B and 2C). However, when the DNA concentration was high, relatively small nanoflower structures having an average size of about 5 μm were formed (FIGS. 2D and 2E). In addition, it could be seen that such nanoflower structures were formed only in the presence of the DNA (FIG. 2F).

### Example 3: Production DNA-Inorganic Hybrid Nanoflowers Using Various DNA Nucleotide Sequences and Lengths

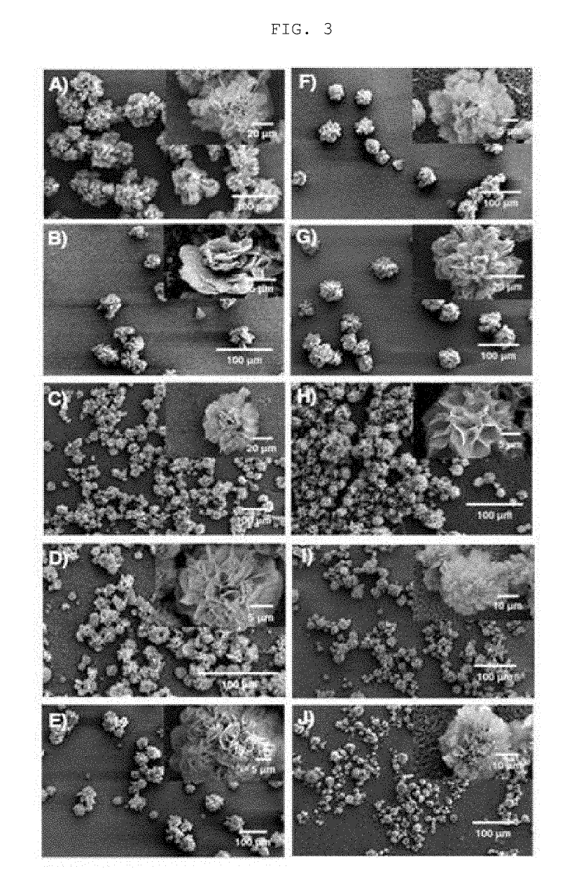

The effect of the DNA nucleotide sequence and length on the production of DNA-inorganic hybrid nanoflowers was examined. FIG. 3 depicts SEM images showing the results of an experiment performed to the effect of the DNA nucleotide sequence and length on the formation of nanoflower structures. Information including the DNA nucleotide sequences used in the experiment is shown in Table 1 below. As can be seen in FIG. 3, all the DNAs (A: dNTPs, B: Adenine-rich single-stranded (ss) DNA, C: Thymine-rich ssDNA, D: Guanine-rich ssDNA, E: Cytosine-rich ssDNA, F: 51-bp Adenine-Thymine double-stranded (ds) DNA, G: 51-bp Guanine-cytosine dsDNA, H: 200-bp PCR amplicon, I: 5420-bp plasmid DNA, J: 4857-kbp genomic DNA) used in the experiment formed flower structures having an average size of 20 to 50 μm, and the DNA encapsulation yield of the produced flower structures was 95% or higher (Table 2). Here, the DNA encapsulation yield is defined as the ratio of the amount of DNA loaded on nanoflower structures to the amount of DNA introduced in the initial stage. In addition, it could be seen that the weight percentage of the loaded DNA in entire nanoflower structures was 7 to 13%, which was similar to that in conventional protein-based flower structures (Table 2, Lin, Y. Xiao et al., ACS. Appl. Mater. Inter., 2014, 6, 10775).

TABLE 1

| DNA samples | Sequences or information |
|---|---|
| A)dNTPs | dATP, dTTP, dGTP and dCTP |
| B)Adenine-rich ssDNA | 5′-AAA AAA AAA AAA TAA AAA AAA AAA |
| | TAAA AAAAAAAAA TAAA AAA AAA AAA-3′ |
| C)Thymine-rich ssDNA | 5′-TTT TTT TTT TTT T TTT TTT TTT TTT T |
| | TTT TTT TT TTT T TTT TTT TTT TTT-3′ |
| D)Guanine-rich ssDNA | 5′-GGG GGG GGG GGG T GGG GGG GGG GGG T |
| | GGG GGG GGG GGG T GGG GGG GGG GGG-3′ |
| E)Cytosine-rich | 5′-CCC CCC CCC CCC TCC CCC CCC CCC TCC |
| ssDNA | CCC CCC CCC TCC CCC CCC CCC-3′ |
| F)ssDNA | 5′-TTT TTT TTT TTT A TTT TTT TTT TTT A |
| complementary to B | TTT TTT T TTT A TTT TTT TTT TTT-3′ |
| for A-T dsDNA | |
| G)ssDNA | 5′-CCC CCC CCC CCC ACC CCC CCC CCC ACC |
| complementary to D | CCC CCC CCC ACC CCC CCC CCC-3′ |
| for G-C dsDNA | |
| H)PCR amplicon (200 | Sample was obtained by amplyfing the |
| bp) | genomic DNA of Chlamy dia trachomatis |
| | using the following primers. |
| | Forward primer: 5′-CTA GGC GTT TGT ACT |
| | CCG TCA-3′ |
| | Reverse primer: 5′-TCC TCA GAA GTT TAT |
| | GCA CT-3′ |
| I)Plasmid DNA (5420 | pETDuet-1 |
| bp) | |
| J)Genomic DNA (4857 | Sample was obtained by purifying the |
| bp) | genomic DNA of Salmonella typhimurium. |

TABLE 2

| | Encapsulation | Weight |
|---|---|---|
| DNA samples | yield (%) | percentage (%) |
| A) dNTPs | 97 | 13 |
| B) Adenine-rich ssDNA | 97 | 10 |
| C) Thymine-rich ssDNA | 99 | 7 |
| D) Guanine-rich ssDNA | 99 | 9 |
| E) Cytosine-rich ssDNA | 99 | 9 |
| F) A-T dsDNA (51 bp) | 98 | 8 |
| G) G-C dsDNA (51 bp) | 98 | 7 |
| H) PCR amplicon (200 bp) | 95 | 10 |
| I) Plasmid DNA (5420 bp) | 99 | 9 |
| J) Genomic DNA (4857 bp) | 97 | 10 |

### Example 4: Examination of Applicability of DNA-Inorganic Hybrid Nanoflowers

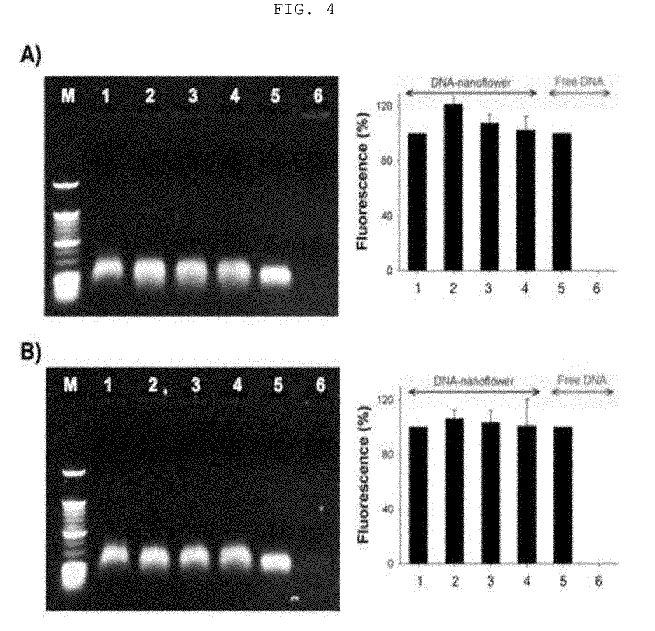

FIG. 4 depicts experimental results indicating that the produced DNA-inorganic hybrid nanoflower structures have resistance against nucleases in an experiment performed to examine the applicability of the DNA-inorganic hybrid nanoflower structures. The experiment was performed to determine whether the DNA loaded on the nanoflower structures would be degraded by typical nucleases, DNase I (FIG. 4A) and exonuclease III (FIG. 4B), and the results of the experiment were confirmed by electrophoresis. As can be seen in FIG. 4, free DNA was completely degraded by the nucleases (lane 5: DNA before reaction; and lane 6: DNA that reacted with DNase I (FIG. 4A) or Exonuclease III (FIG. 4B) for 30 minutes). However, it could be seen that the DNA loaded on the nanoflower structures had resistance against the two kinds of nucleases and stably maintained its structure even after 24 hours of a sufficient enzymatic reaction (lane 1: DNA-inorganic hybrid nanoflower structures before reaction, lane 2: DNA-inorganic hybrid nanoflower structures reacted with DNAse I (A) or Exonuclease III (B) enzyme for 30 mins, lane 3: DNA-inorganic hybrid nanoflower structures reacted with DNAse I (A) or Exonuclease III (B) enzyme for 6 hrs, lane 4: DNA-inorganic hybrid nanoflower structures reacted with DNAse I (A) or Exonuclease III (B) enzyme for 24 hrs).

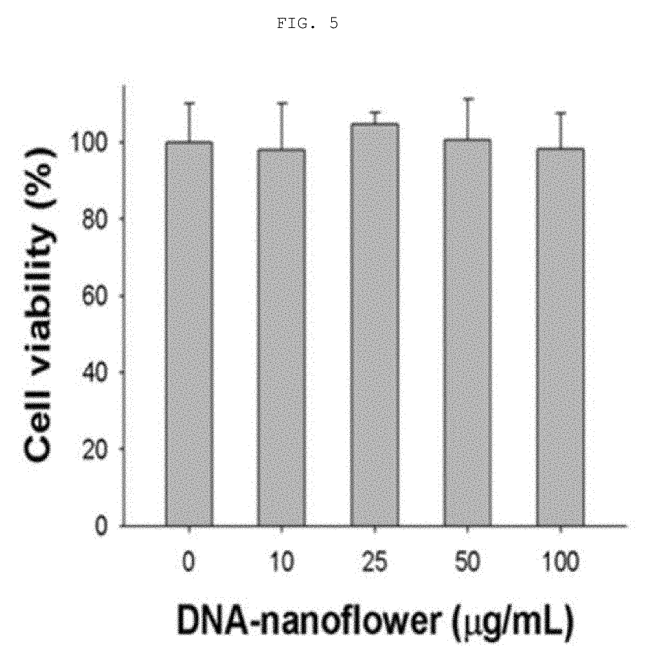

In addition, various concentrations of the DNA-inorganic hybrid nanoflower structures were introduced into cells which were then incubated for 24 hours, after which the effect of the toxicity of the nanoflower structures on the cells was analyzed. The results of the analysis are shown in FIG. 5. As shown in FIG. 5, it could be seen that the DNA-inorganic hybrid nanoflower structures had no cytotoxicity (100% cell viability). Based on such excellent characteristics, the DNA-inorganic hybrid nanoflower structures are expected to be utilized as a carrier for gene therapy in the future.

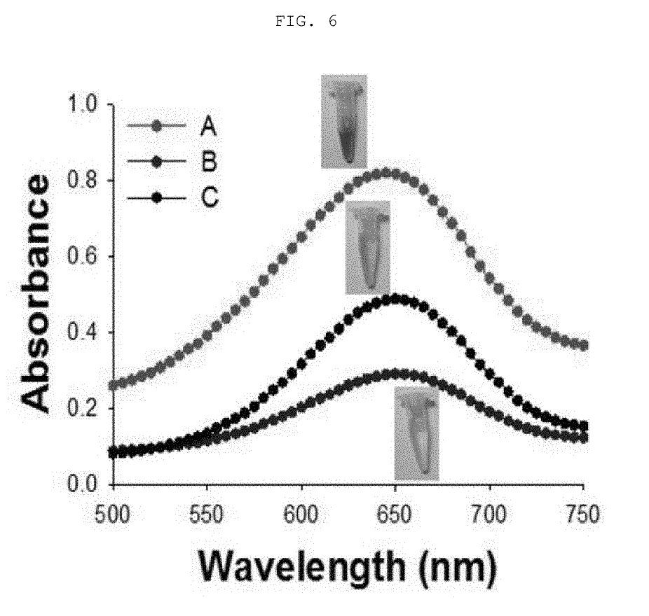

Furthermore, the peroxidase activity of the synthesized DNA-inorganic hybrid nanoflower structures was analyzed, and the results of the analysis are shown in FIG. 6. As can be seen in FIG. 6, when DNA was absent, a precipitate formed from the copper sulfate salt showed a very low peroxidase activity (FIG. 6B). However, it could be seen that the nanoflower structures synthesized by the reaction between the DNA and the copper sulfate salt showed high peroxidase activity due to their large surface area (FIG. 6A). Furthermore, it was found that the DNA-inorganic hybrid nanoflower structures had higher peroxidase activity than that of conventional protein-based nanoflower structures. Thus, the DNA-inorganic hybrid nanoflower structures synthesized according to the present invention is expected to be widely utilized in biosensing technology for high-sensitivity detection of target biomaterials in the future.

### INDUSTRIAL APPLICABILITY

The method of preparing nucleic acid-inorganic hybrid nanoflowers according to the present invention has an effect in that nucleic acid-inorganic hybrid nanoflowers can be synthesized in a very simple manner using the nucleic acid at room temperature under an environmentally friendly condition without addition of any toxic reducing agents or the like. The nucleic acid-inorganic hybrid nanoflowers thus produced have low cytotoxicity, show significantly increased loading capacities of 95% or more compared to those produced by a conventional DNA loading technology, and have high resistance against nuclease, so that they can have high utilization value as a carrier for gene therapy and a biosensor for high-sensitivity detection of target biomaterials.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A method of preparing nucleic acid-inorganic hybrid nanoflowers, comprising forming a complex between a metal ion and a nitrogen atom of an amide bond or amine group in the nucleic acid, by reacting the nucleic acid with a solution of the metal ion-containing compound at room temperature.

2. The method of preparing nucleic acid-inorganic hybrid nanoflowers of claim 1, wherein the nucleic acid is DNA or RNA.

3. The method of preparing nucleic acid-inorganic hybrid nanoflowers of claim 1, wherein the metal is at least one selected from the group consisting of copper (Cu), zinc (Zn), calcium (Ca) and manganese (Mn).

4. The method of preparing nucleic acid-inorganic hybrid nanoflowers of claim 1, wherein the metal ion-containing compound is at least one selected from the group consisting of copper sulfate (CuSO₄), zinc acetate (Zn(CH₂COO)₂), calcium chloride (CaCl₂)) and manganese sulfate (MnSO₄).

5. The method of preparing nucleic acid-inorganic hybrid nanoflowers of claim 1, wherein the reaction is performed at room temperature for 60 to 80 hours.

6. The method of preparing nucleic acid-inorganic hybrid nanoflowers of claim 1, wherein a concentration of the nucleic acid is 10 pM to 1 μM according to a length of base sequence.

7. The method of preparing nucleic acid-inorganic hybrid nanoflowers of claim 1, wherein the size of the nucleic acid-inorganic hybrid nanoflowers is determined depending on a concentration of the nucleic acid.

8. Nucleic acid-inorganic hybrid nanoflowers having resistance against nuclease, which are produced by the method of claim 1.

9. The nucleic acid-inorganic hybrid nanoflowers of claim 8, wherein a weight percentage of the nucleic acid in total nanoflowers is 7 to 13 wt %.

10. A carrier for gene therapy, which comprises the nucleic acid-inorganic hybrid nanoflowers of claim 8.

11. A biosensor comprising the nucleic acid-inorganic hybrid nanoflowers of claim 8.


SEQUENCE LISTING (8 sequences)

SEQ ID NO: 1 - DNA, 51 bp, Artificial Sequence
  Adenine-rich ssDNA
aaaaaaaaaaaataaaaaaaaaaaataaaaaaaaaaaataaaaaaaaaaaa

SEQ ID NO: 2 - DNA, 50 bp, Artificial Sequence
  Thymine-rich ssDNA
tttttttttttttttttttttttttttttttttttttttttttttttttt

SEQ ID NO: 3 - DNA, 51 bp, Artificial Sequence
  Guanine-rich ssDNA
ggggggggggggtggggggggggggtggggggggggggtgggggggggggg

SEQ ID NO: 4 - DNA, 48 bp, Artificial Sequence
  Cytosine-rich ssDNA
cccccccccccctccccccccccctccccccccccctccccccccccc

SEQ ID NO: 5 - DNA, 49 bp, Artificial Sequence
  ssDNA complementary to SEQ ID NO 1
ttttttttttttattttttttttttattttttttttatttttttttttt

SEQ ID NO: 6 - DNA, 48 bp, Artificial Sequence
  ssDNA complementary to SEQ ID NO 3
ccccccccccccacccccccccccacccccccccccaccccccccccc

SEQ ID NO: 7 - DNA, 21 bp, Artificial Sequence
  Forward primer
ctaggcgtttgtactccgtca

SEQ ID NO: 8 - DNA, 20 bp, Artificial Sequence
  Reverse primer
tcctcagaagtttatgcact